(19) United States
(12) Patent Application Publication
(10) Pub. No.: US 2018/0286502 A1
(43) Pub. Date: Oct. 4, 2018

(54) PATIENT DATA MANAGEMENT

(71) Applicant: Welch Allyn, Inc., Skaneateles Falls, NY (US)

(72) Inventors: John A. Lane, Weedsport, NY (US); Matthew J. Kinsley, Liverpool, NY (US); David J. Rider, Baldwinsville, NY (US)

(21) Appl. No.: 15/937,090

(22) Filed: Mar. 27, 2018

Related U.S. Application Data
(60) Provisional application No. 62/480,605, filed on Apr. 3, 2017.

(51) Int. Cl. G16H 10/60; G16H 50/20; G16H 20/00

(52) U.S. Cl. CPC G16H 10/60; G16H 20/00; G16H 50/20

(57) ABSTRACT

Example systems and methods are provided to effectively manage a large number of patients by prioritizing treatments to the patients. The example system provides the triage of multiple patient data with weightings. The system further provides a treatment regimen suitable for a diagnosed disease and an incentive option for a healthcare practitioner that enrolls a patient to the regimen and the patient who complies with the regimen.

### BACKGROUND

Patient-generated health data (PGHD) is health-related data created, recorded, or gathered by or from patients, family members, or other caregivers to help address a health concern. Healthcare practitioners are increasingly inundated with patients wanting to share this data. There is need to effectively review such data in concert with other key information to make effective treatment decisions.

### SUMMARY

In general terms, this disclosure is directed to patient data management systems and methods. In one possible configuration and by non-limiting example, an example system or method can effectively manage a large number of patients by prioritizing treatments to the patients. Various aspects are described in this disclosure, which include, but are not limited to, the following aspects.

One aspect is a method for providing health treatment to a patient, comprising: receiving, at a computing device, regimen data, the regimen data including a plurality of regimens associated with a plurality of diseases; receiving, at the computing device, incentive data, the incentive data including a plurality of incentive options associated with the plurality of regimens; obtaining a diagnosis of a disease; selecting, using the computing device, a regimen from the regimen data based on the diagnosis; selecting, using the computing device, an incentive option from the incentive data based on the determined regimen; determining, using the computing device, compliance of the regimen; and providing one or more incentives to the patient, the incentives identified from the incentive option.

Another aspect is a method for managing patient data, comprising: receiving, at a computing device, patient data including a plurality of patient data elements; setting, using the computing device, one or more thresholds on at least one of the patient data elements; assigning, using the computing device, one or more weightings on at least two of the patient data elements; and flagging, using the computing device, the patient data elements based on the thresholds and the weightings.

### BRIEF DESCRIPTION OF THE DRAWINGS

FIG. 1 illustrates an example system for managing patient data.

FIG. 2A illustrates an example of assigning thresholds to patient health data.

FIG. 2B illustrates an example of assigning weighting for the patient health data as shown in FIG. 2A.

FIG. 3 illustrates other aspects of the system for managing patient data.

FIG. 4 illustrates an example regimen evaluation data.

FIG. 5 illustrates an example method for managing patient data and providing incentives.

FIG. 6 illustrates an exemplary architecture of a computing device which can be used to implement aspects of the present disclosure.

### DETAILED DESCRIPTION

Various embodiments will be described in detail with reference to the drawings, wherein like reference numerals represent like parts and assemblies throughout the several views.

In general, the present disclosure provides a system for effectively managing a large number of patients by prioritizing treatments to the patients. The system can enable healthcare practitioners to set thresholds on individual or combined patient data elements, such as patient-generated health data. The system can flag the patient data elements based on the thresholds. The system can further allow weighting the patient data elements differently to improve patient data management.

In certain examples, the system of the present disclosure provides the triage of multiple patient data with weightings. The weightings can be controlled by healthcare practitioners, such as clinicians for patients. The system may provide an interface for healthcare practitioners to set or adjust weighting for the patient data elements. A healthcare practitioner can further set a threshold for flagging patient data.

Further, the system of the present disclosure provides a treatment regimen suitable for a diagnosed disease and an incentive option for a healthcare practitioner that enrolls a patient to the regimen and the patient who complies with the regimen.

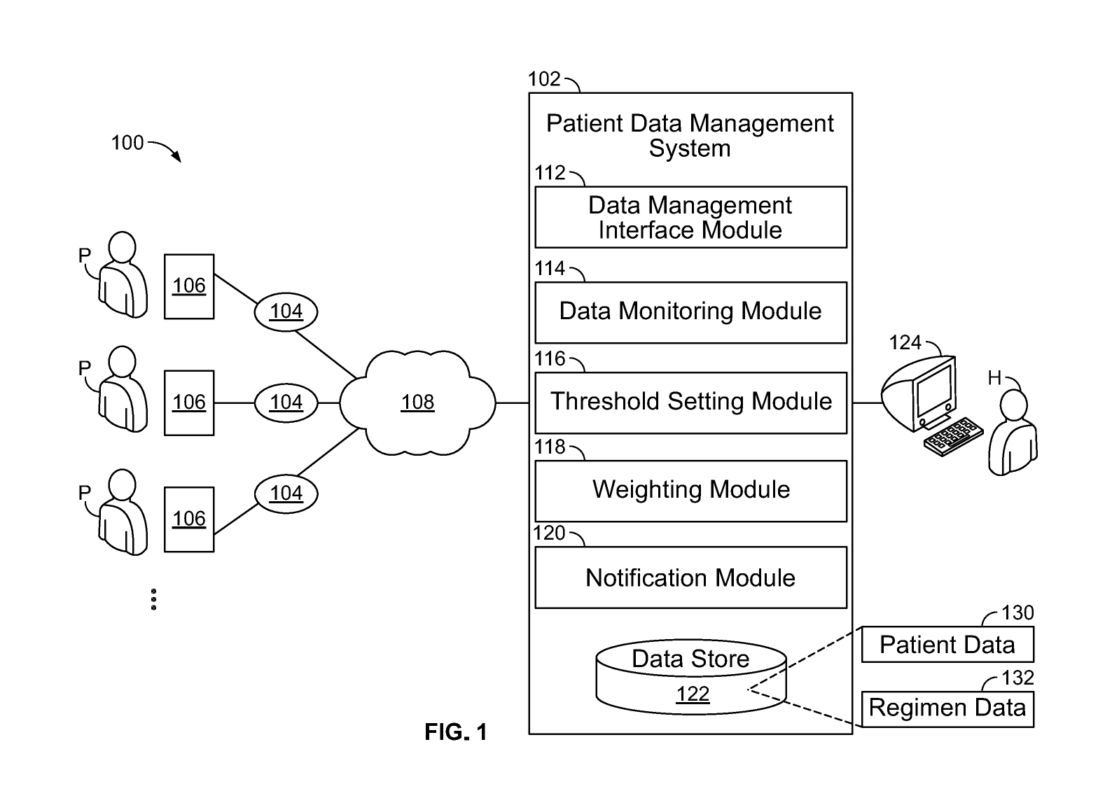

FIG. 1 illustrates an example system 100 for managing patient data. The system 100 includes a patient data management system 102 for receiving and managing patient-generated data. The patient data management system 102 can receive patient health data 104 from a plurality of patient computing devices 106 operated by patients P. The patient data management system 102 operates to process, evaluate, and manage the received patient health data 104, and further generates data useful for a healthcare practitioner H to monitor the patient health data 104 and provide desirable service to the patients P. An example of the patient data management system 102 is further described herein.

In some embodiments, one or more patients P use patient computing devices 106 to measure the patient's physiological parameters and generate patient health data 104. The patient health data 104 include information about the measured physiological parameters that can represent the patient's health condition. In general, physiological parameters can include vital signs, physiological measurements, and biological measurements, which can be detected from various portions of the patient's body.

For example, physiological parameters include measurements of the body's basic functions, which are useful in detecting or monitoring medical problems. Examples of physiological parameters include body temperature, pulse rate (i.e., heart rate), respiration rate (i.e., breathing rate), blood pressure, blood gas, and SpO2. Typically, body temperature can be taken in various manners, such as orally, rectally, by ear, or by skin. The pulse rate is a measurement of the heart rate, or the number of times the heart beats per minute. The pulse rate can also indicate a heart rhythm and the strength of the pulse. The pulse can be taken on different body portions where the arteries are located, such as on the side of the neck, on the side of the elbow, or at the wrist. The respiration rate is the number of breaths a person takes per minute and is used to note whether the person has any difficulty breathing. Blood pressure is the force of the pushing against the artery walls.

There may be other vital signs, such as weight, oximetry, pain, Glasgow coma scale, pulse oximetry, blood glucose level, end-tidal CO₂, functional status, shortness of breath, gait speed, and other vitals.

The patient computing device 106 can be of various configurations. In some embodiments, the patient computing device 106 is configured in the form of, for example, a mobile phone, a tablet computer, an internet enabled television, an internet enabled gaming system, a desktop, and other computing devices. Alternatively or in addition, the patient computing device 106 includes a medical device of various types, which can depend on the patient's chronic diseases to be cared for.

For example, where diabetes is concerned, the patient medical device can be a blood glucose meter or other device for measuring blood glucose. In other examples, the medical device can be an insulin injection device (e.g., an insulin pen) for diabetes; an artificial pancreas device for diabetes; a vest machine for patients with cystic fibrosis; a nebulizer or an inhaler for patients with cystic fibrosis, asthma, COPD, and other respiratory diseases, or a device that measure hemoglobin. Other types of medical device are also possible.

In some embodiments, such a medical device can be incorporated in another computing device, such as the patient's mobile phone, tablet computer, internet enabled television, internet enabled gaming system, desktop, and other computing devices. In other embodiment, the medical device can be formed independently from such other computing devices.

The patient computing devices 106 can communicate with the patient data management system 102 over a data communication network 108. The data communication network 108 communicates digital data between one or more computing devices, such as among the patient data management system 102, the patient computing devices 106, and the practitioner computing devices 124. Examples of the network 108 include a local area network and a wide area network, such as the Internet. In some embodiments, the network 108 includes a wireless communication system, a wired communication system, or a combination of wireless and wired communication systems. A wired communication system can transmit data using electrical or optical signals in various possible embodiments.

Wireless communication systems typically transmit signals via electromagnetic waves, such as in the form of optical signals or radio frequency (RF) signals. A wireless communication system typically includes an optical or RF transmitter for transmitting optical or RF signals, and an optical or RF receiver for receiving optical or RF signals. Examples of wireless communication systems include Wi-Fi communication devices (such as utilizing wireless routers or wireless access points), cellular communication devices (such as utilizing one or more cellular base stations), and other wireless communication devices.

Referring still to FIG. 1, the patient data management system 102 includes a data management interface module 112, a data monitoring module 114, a threshold setting module 116, a weighting module 118, a notification module 120, and a data store 122.

The data management interface module 112 is configured to receive the patient health data 104 from the patient computing devices 106 via the network 108. Further, the data management interface module 112 enables the patient data management system 102 to connect to a computing device 124 operated by a healthcare practitioner H.

In some embodiments, the practitioner computing device 124 is connected to the patient data management system 102 via the network 108 or another network. In other embodiments, the practitioner computing device 124 includes or runs the patient data management system 102. The healthcare practitioner H can control and operate the system 102 via the practitioner computing device 124, and retrieve data from the system 102 via the practitioner computing device 124.

The data monitoring module 114 operates to monitor the patient health data 104. In some embodiments, the data monitoring module 114 can monitor the patient health data 104 of a particular type for a particular patient over a predetermined period of time. The data monitoring module 114 then can obtain a trend of the patient health data 104 of that type for the patient. Such a trend of patient health data can be used to evaluate the health condition of the subject patient, as further discussed herein.

In some embodiments, the patient health data 104 which can be monitored by the data monitoring module 114 include various physiological parameter readings as described herein. In other embodiments, the patient health data 104 include the trend of such readings and the average of readings over a period of time can also be determined by the data monitoring module 114.

In yet other embodiments, the data monitoring module 114 can detect patient's compliance or adherence with prescribed treatment regimens, such as medications compliance or testing schedule compliance. Such treatment regimens can be provided to patients who suffer from chronic diseases to manage or improve the chronic diseases. Treatment regimens may require patients to take actions on a regular basis. For example, some treatment regimens for managing the effects of diabetes provide for monitoring a diabetic's blood glucose level by using a blood glucose meter to periodically obtain a blood glucose reading from the diabetic's blood. Such treatment regimens may include a schedule by which blood glucose readings should be taken, and/or an acceptable range within which a blood glucose level should fall. In some embodiments, the healthcare provider H can set up treatment regimens for managing patients' conditions, such as a chronic disease.

As described herein, some embodiments of a treatment plan include schedules for blood glucose monitoring, dietary plans, exercise plan, and medications. In examples, the treatment regimen includes glucose testing at a predetermined times, taking medications including insulin at predetermined times or in response to blood sugar levels, and eating at predetermined schedules or in response to blood glucose levels.

The threshold setting module 116 operates to set thresholds on data elements of the patient health data 104. In some embodiments, a threshold can be associated with individual data elements. In other embodiments, a threshold can be associated with a group of data elements. As described herein, patient data elements can be flagged based on the thresholds.

In some embodiments, the threshold setting module 116 enables the healthcare practitioner to input threshold information for associated patient health data via the practitioner computing device 124 (e.g., an input device thereof). By way of example, a threshold for a blood pressure can be set such that a predetermined percentage of increase in blood pressure readings over a predetermined period of time triggers a flag for a patient having such blood pressure readings.

The weighting module 118 operates to set weighting on the patient data elements for evaluation of the patient health data 104. The weightings can be controlled by healthcare practitioners. In some embodiments, the weighting module 118 provides a user interface for the healthcare practitioner to set or adjust weighting for the patient data elements.

By way of example, a threshold for a blood pressure can be set such that a predetermined percentage of increase in blood pressure readings over a predetermined period of time triggers a flag for a patient having such blood pressure readings. If the healthcare practitioner sets 100% weighting on blood pressure trending, only the patient's blood pressure readings are considered and any other patient data will not be counted. In this example, the patient will be flagged when the patient's blood pressure readings exceed the preset threshold. When the patient data is flagged, the healthcare practitioner can review the data and see the patient if necessary.

The notification module 120 operates to generate a notification to the healthcare practitioner. The notification can include various pieces of information regarding the patient health data 104 and the evaluation thereof. In some embodiments, the notification can include flagging information based on the monitoring of the patient health data. As described herein, for example, a patient or patient health data associated with the patient can be flagged when the patient's blood pressure readings exceed a preset threshold. When the patient data is flagged, the healthcare practitioner can review the health data for further evaluation. The notification can be of various formats, such as visual and audible, and can be provided through the practitioner computing device 124.

The data store 122 stores various data including patient data 130 and regimen data 132. The patient data 130 include the patient health data 104 received from the patient computing devices 106 and other data resulting from process or evaluation of the patient health data 104. In some embodiments, the patient data 130 include patient identification information, biographical information, and physiological parameter readings as described herein. The regimen data 132 include information about treatment regimens created by the healthcare practitioner for patients.

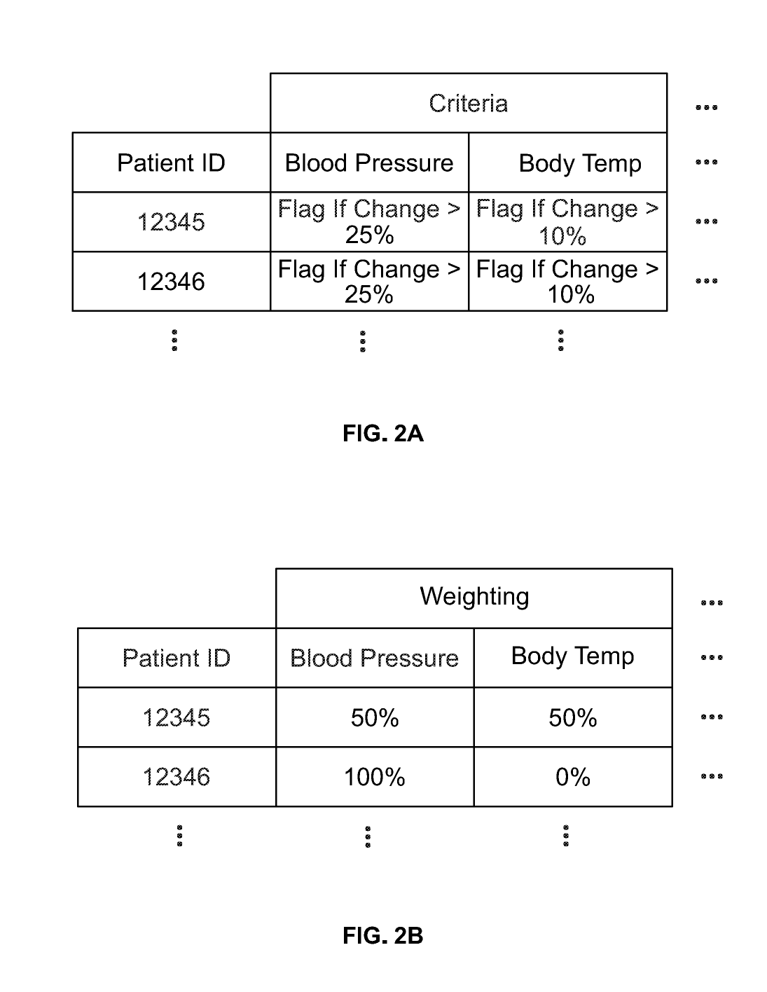

FIG. 2A illustrates an example of assigning thresholds to patient health data, and FIG. 2B illustrates an example of assigning weighting for the patient health data as shown in FIG. 2A.

Referring to FIG. 2A, each patient can be identified with a patient identification (ID). In this example, each patient is monitored for patient health data elements including a blood pressure, a body temperature, and other conditions. Criteria include thresholds which are associated with the patient health data elements. In this example, a threshold for the blood pressure readings of a first patient (Patient ID: 12345) is set such that, if a change in the blood pressure readings over a predetermined period of time (or between two preset times) exceeds 25%, the first patient is flagged. In some embodiments, thresholds can be set for individual patients. In other embodiments, a threshold can be identically set for a plurality of patients or all of the patients in a predetermined group.

Referring to FIG. 2B, weighting can be set for patient health data elements. In this example, for the first patient (Patient ID: 12345), the blood pressure monitoring and the body temperature reading are equally weighted (i.e., 50% for each). For a second patient (Patient ID: 12346), the blood pressure monitoring is weighted 100% while the body temperature (as well as other patient data elements) are not considered (i.e., 0% weighted). In some embodiments, weighting can be set for individual patients. In other embodiments, weighting can be identically set for a plurality of patients or all of the patients in a predetermined group.

A more thorough example can include many selected inputs. For example, various inputs, such as increase in weight (e.g., >5%), medication compliance (e.g., yes/no), any heart rate readings (e.g., over 170/100), and dosage of medications, can be considered. The healthcare practitioner can assign a weighting of these aspects depending on the patients situation.

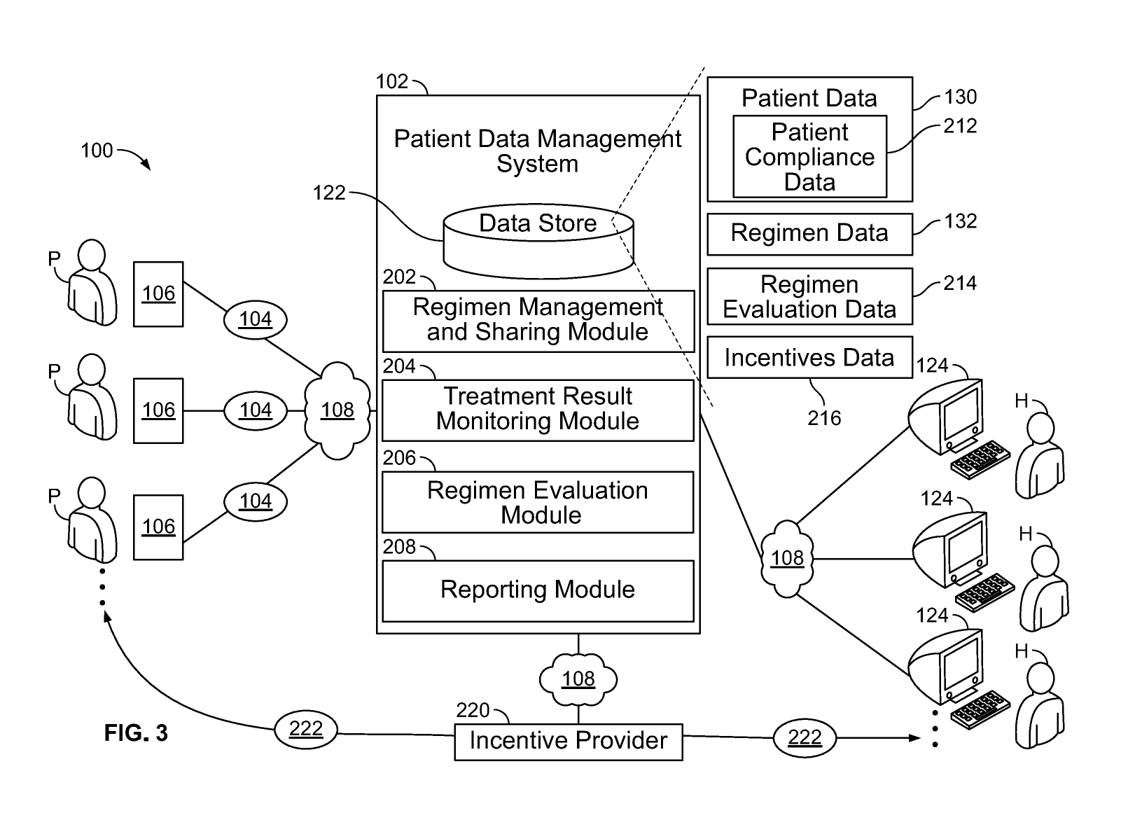

FIG. 3 illustrates other aspects of the system 100 for managing patient data. In addition or alternatively, the patient data management system 102 includes a regimen management and sharing module 202, a treatment result monitoring module 204, a regimen evaluation module 206, and a reporting module 208. In this example, a plurality of healthcare practitioners H can be involved.

The regimen management and sharing module 202 is configured to manage a plurality of treatment regimens which is created, revised, and updated by a plurality of healthcare practitioners H. The treatment regimens are stored as regimen data 132 in the data store 122. The module 202 is further configured to enable the healthcare practitioners H to share their regimens with other healthcare practitioners H. In some embodiments, the module 202 can operate to provide the healthcare practitioners H with different levels of access to the regimen data 132. The module 202 allows the healthcare practitioners to search for treatment regimens created by other practitioners and modify the regimens to suit their patients.

By way of example, a primary care doctor in one place creates a hypertension management regimen that the doctor uses on his or her patients. For example, the regimen requires the patient to take a blood pressure measurement once a day, take a weight measurement once a day, use an ACE inhibitor, and add three days of exercise. This regimen might be able to reduce systolic averages by 7%. Another primary doctor in another place creates a hypertension management regimen that requires the patient to measure blood pressure and weight once every other day at specific times, use Alpha blockers, and walk one mile every other day with a pedometer. This practice might be able to reduce clinical visits by 10%. These two example regimens can be shared with other healthcare practitioners through the system 102.

In some embodiments, some standard patient protocols or regimens can be initially provided for healthcare practitioner side data triage and flag process. Flagging or notification to healthcare practitioners can occur under predetermined circumstances. The flagging or notification can be of various formats, such as texting, telephone calls, emailing, or marking of the patient in the database. The flagging or notification can also be delivered to the patient to set a follow-up appointment.

By way of example, one treatment regimen or protocol is set such that measurements are taken once in the morning and once in the afternoon every day and flagged if the trend of readings changes certain percentage (e.g., 25%) less or greater than the overall average for a predetermined period of time (e.g., 5 days), if the blood pressure is greater than a predetermined number (e.g., 160/80), and/or if constant motion error or equipment failure is detected.

Another treatment regimen or protocol can be associated with patient compliance and is set such that measurements are taken once every day at a particular time (e.g., noon) and flagged if the medications change within a predetermined period of time (e.g., last 30 days), if the patient fails to comply with taking measurements, if medications are not taken as planned or the patient refuses medications, and/or if potential interaction with other medications are determined.

Yet another treatment regimen or protocol can be associated with vital signs and is set such that measurements are taken once every other day and is flagged if a combination of vital signs (e.g., a combination of blood pressure change, weight change, and temperature change) changes greater than a predetermined percentage, if arrhythmia is detected, and/or if a recent ER visit is detected.

The healthcare practitioners can selectively edit and customize the existing regimens. The healthcare practitioners can also mix the attributes of the regimens. In some embodiments, the system 102 can use the population health data to optimize the regimens, or provide optimal regimens to the healthcare practitioners. The system 102 can also monitor how the regimens meet target goals for the patient population handled by the healthcare practitioner. By way of example, such a target goal can be that all patients should be under 160/80 heart rate with medications or that the overall patient base is reduced 20% lower. As described herein, regimens can be tied to disease states. In addition or alternatively, regimens can be tied to individual or groups of other physiological parameters.

The system 102 can also operate to enable patients to leave notes in the database and allow the healthcare practitioners to review the notes. If survey information from the patient can cause concern, such as privacy issue on patient's mental health issue, the healthcare practitioners can be flagged. The system 102 can also be configured to guide the healthcare practitioners to set up the database and identify overall goals for the entire patient population.

The treatment result monitoring module 204 operates to receive and monitor the patient health data 104 from the patients P. In some embodiments, the module 204 can determine the trend of the patient health data 104 for a particular patient and/or a particular type of disease. As described herein, the patient health data 104 can include the measurements or readings of one or more physiological parameters (such as blood pressure, glucose level, etc., as described herein) of patients that are obtained using the patient's medical devices, which can be associated with or incorporated in the patient computing devices 106.

In addition, the patient health data 104 can include patient compliance data 212 which can indicate compliance status of the patients with the treatment regimens prescribed to them. In some embodiments, the patient compliance data 212 can include information whether or not the patients complied with their treatment regimens. In other embodiments, the patient compliance data 212 can show how well the patients adhered to the treatment regimens.

In some embodiments, the patient compliance data 212 can be obtained by monitoring whether the patients have timely performed required measurements over a period of time. In addition or alternatively, the patient compliance data 212 can be obtained by monitoring whether the patients have timely taken required medications over a period of time. Other methods for obtaining the patient compliance data are also possible.

The regimen evaluation module 206 operates to evaluate the treatment regimens. In some embodiments, the module 206 determines how well the regimens have performed with the patients associated with the regimens. In some embodiments, the regimen evaluation module 206 can generate regimen evaluation data 214 based on the evaluation of the treatment regimens. The regimen evaluation data 214 can provide information about how much the regimens improve the patients' health conditions. By way of example, the regimen evaluation data 214 can indicate what percentage a particular regimen improves a patient's hypertension.

In some embodiments, the regimen evaluation data 214 can be used to determine incentives 222 for the healthcare practitioners and/or patients. For example, if the regimen evaluation data 214 shows that a particular regimen has improved the treatment of a group of patients, incentives can be provided to the patients who complied with the regimen and/or the healthcare practitioners who created the regimen or prescribed the regimen to the patients. The incentives can be determined and provided by incentive providers 220, such as private or public healthcare providers or companies (e.g. Medicaid and health insurance companies)

Various incentives can be provided, such as financial benefit, reimbursement, discount, and any suitable form of incentive. Such incentive options can be stored in incentive data 216. The incentive data 216 can include, for example, patient types (types of diagnoses or diseases), incentivized regimens, types of incentives, criteria for triggering incentives, and any suitable data. In some embodiments, the incentive data 216 can be stored in the data store 122 associated with the patient data management system 102. In other embodiments, the incentive data 216 can be stored in another database which is accessible by the incentive providers 220.

The reporting module 208 is configured to generate a report to the healthcare practitioners, the patients, and other interested parties, such as public or private insurance providers or companies. The report includes various pieces of information, such as the summary of the information (including the patient data 130, the regimen data 132, the regimen evaluation data 214, and the incentives data 216) obtainable from the modules 202, 204, and 206. The report can be presented (e.g., displayed) via computing devices, or produced as printouts using printing devices. The report is customizable by, for example, the healthcare practitioners, patients, or incentive providers.

The networks 108 illustrated in FIG. 3 can be the same network in some embodiments or different networks in other embodiments.

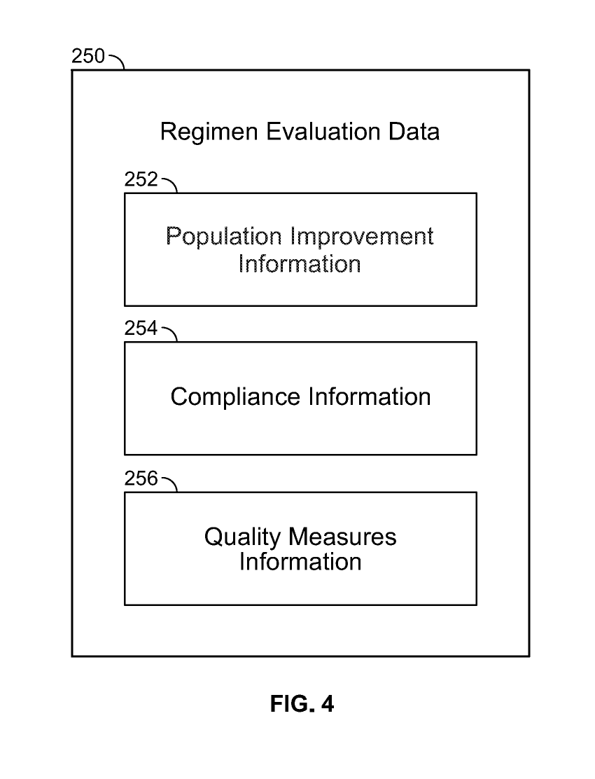

FIG. 4 illustrates an example regimen evaluation data 250, which can be generated by the regimen evaluation module 206 in FIG. 3. The regimen evaluation data 250 includes various pieces of information about the regimens, such as performance evaluation of the regimens that have been created and used as illustrated in FIG. 3. The regimen evaluation data 250 can further be used by healthcare practitioners to revise and update the existing regimens to achieve improved results. In some embodiments, the regimen evaluation data 250 includes population management information 252, compliance information 254, and quality measures information 256.

The population management information 252 includes improvement for a population group as a whole from particular regimens. The population group can be determined in various manners based on various factors, such as size, age, race, gender, etc. In some embodiments, the population management information 252 can be used to efficiently manage other data, such as the patient data 130 and the regimen data 132. In other embodiments, the population management information 252 can be used to determine incentives, such as financial benefits, to the healthcare practitioners that are associated with the regimens in question. For example, the healthcare practitioners that have created or used one or more regimens which lead to improvement in a population group (e.g., reduction in hypertension readings or risks for certain patients) can receive additional payments or increased reimbursement under one or more associated insurance programs, such as private health insurance programs or public health insurance programs. Insurance companies incentivize developing and promoting regimens that reduce healthcare costs and improve efficiency, such as reducing unnecessary patient visits.

In addition, the population management information 252 can include a routine trending summary that can drive incentives for the practice of one or more corresponding regimens.

The compliance information 254 provides compliance summary so that healthcare practitioners can determine how many of the patients have been following a particular regimen and/or how well the patients followed the regimen. The compliance summary can be used for increased reimbursement to healthcare practitioners (e.g., doctors, clinicians, clinics, hospitals, etc.) and insurance providers. In some embodiments, regimens can drive higher compliance. In other embodiments, regimens can work for third party incentive payers, such as workplace health management systems. In certain examples, some patients are obligated to follow certain regimens, such as workplace health management programs. For example, when patients are truck drivers, some of them are not allowed to have certain class of license (e.g., Class A CDL license) or medical transport if they are hypertensive. In other examples, workers in public transportation departments must be periodically cleared in certain states.

The quality measures information 256 provides a summary of population effectiveness of regimens, regimen performance, quality of patient care, and any other suitable information. In some embodiments, the summary of the quality measures information 256 is customizable.

In some embodiments, the regimen evaluation data 250 is included in a report generated by the reporting module 208.

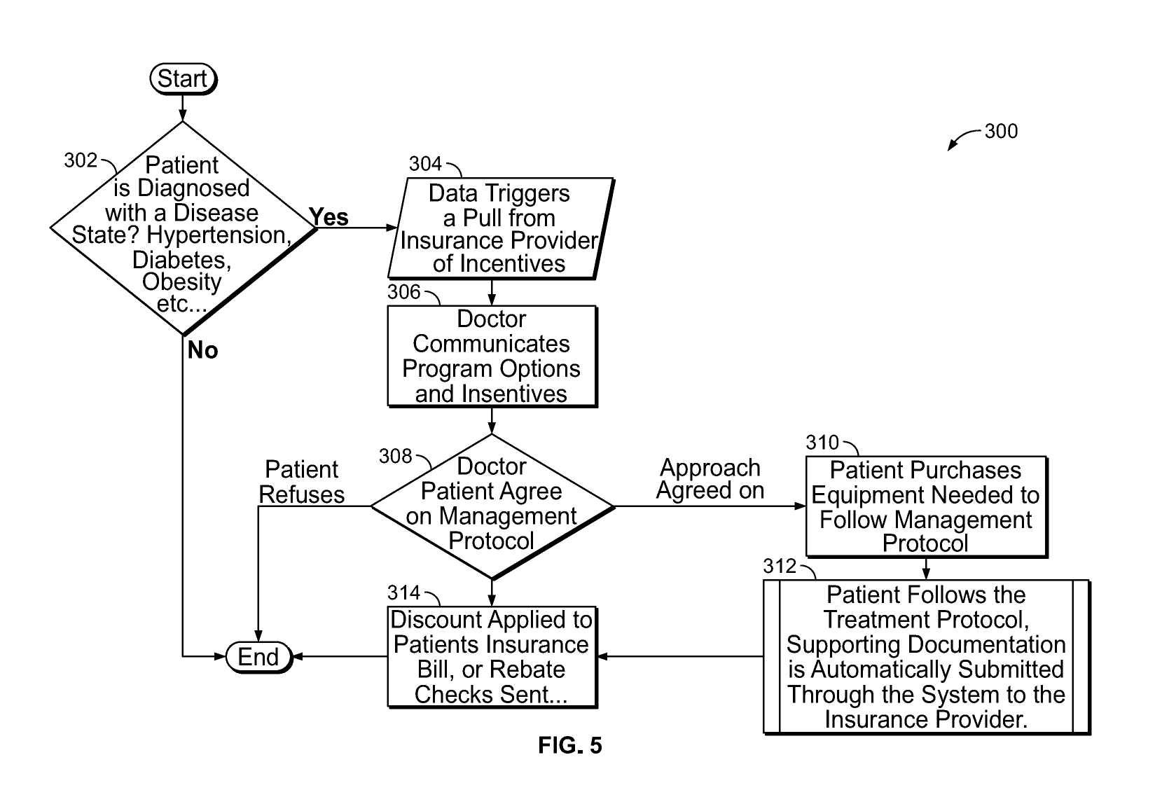

FIG. 5 illustrates an example method 300 for managing patient data and providing incentives. As illustrated, at operation 302, it is first determined whether a patient is diagnosed with a disease. For example, a healthcare practitioner, such as a clinician, can diagnose the patient.

Once a disease is identified, the information about the disease can be provided to the patient data management system 102. At operation 304, the patient data management system 102 operates to retrieve the regimen data 132 and the incentives data 216. The regimen data 132 and the incentives data 216 can be used to determine one or more suitable regimens for the patient and/or the disease and incentives associated with the regimen. By way of example, the regimen data 132 and/or the incentives data 216 can be managed by the incentive provider 220, such as an insurance provider, and the incentive provider 220 identifies a regimen for the diagnosed disease and an incentive for the regimen.

In some embodiments, the incentive provider 220 has triggers for predetermined diseases. Such predetermined diseases can include diseases requiring increased medical costs and imposing financial burden on the incentive provider 220. The predetermined diseases can be associated with one or more regimens for effectively and efficiently treating the diseases, and such regimens can be tied to incentives for healthcare practitioners and patients.

At operation 306, once the suitable regimens and incentives are identified for the patient who is diagnosed with the disease, the regimens and incentives can be communicated to the patient. For example, the healthcare practitioner who cares for the patient can inform the patient of the available regimens and incentives.

In some examples, the healthcare practitioner can receive one or more incentives, such as financial benefit, from the incentive provider 220 when the healthcare practitioner enrolls the patient in the regimens determined by the patient data management system 102. In addition or alternatively, the patient can receive one or more incentives, such as financial benefit, when the patient follows the prescribed regimens as instructed.

Different incentives can be provided for different regimens. In some embodiments, more aggressive regimens can be incentivized higher because such regimens have greater health improvement potential.

At operation 308, the healthcare practitioner determines that the regimen obtained from the system 102 is suitable for the patient. If the patient agrees with the practitioner's determination, the method 300 continues at operation 310. Otherwise, the method 300 ends.

At operation 310, if the regimen requires equipment, such as a medical device for measuring physiological attributes, the patient can purchase the equipment to comply with the regimen. Such equipment may or may not be reimbursable by the incentive provider. As described herein, the healthcare practitioner who enrolls the patient to the regimen can receive incentives (e.g., payment) for the patient's enrollment and/or purchase of required equipment.

At operation 312, when the patient complies with the regimen, such compliance can be communicated to the system 102. In some embodiments, the patient's computing device or medical device communicates with the system 102 and transmits measurement data to the system 102, and the system 102 can determine whether the patient has been adhere to the regimen based on the measurement data (such as the patient health data 104). In other embodiments, the patient or the healthcare practitioner can provide other information to prove the patient's compliance with the regimen. For example, supporting documentation can be submitted to the system 102 or other computing devices so that the incentive provider 220 can review the documentation.

At operation 314, the incentives associated with the regimen are provided to the patient and/or the healthcare practitioner. For example, various incentives, such as discounts on insurance premium and rebate checks, are provided to the patient who is determined to have adhered to the prescribed regimen.

As described herein, the method of the present disclosure provides a compensation model for physicians (as well as patients and other entities) on clinical and cost-saving outcomes rather than being reimbursed for services and procedures. Because primary care physicians help more patients with managing chronic illnesses, metrics such as reducing hospital readmissions, unneeded diagnostic test, and showing more efficient care essentially reward physicians who can keep track of how they keep patient care cost-effective.

In some embodiments, the system and method of the present disclosure is applicable to various value-based healthcare service programs or models, such as the Patient-Centered Medical Home (PCMH) models, the Physician Quality Reporting System (PQRS) and the Value-Based Payment Modifier offered by the Centers for Medicare and Medicaid Services (CMS). The Patient-Centered Medical Home (PCMH) model is a care delivery model whereby patient treatment is coordinated through their primary care physician to ensure they receive the necessary care when and where they need it, in a manner they can understand. The Physician Quality Reporting System (PQRS) is a quality reporting program that encourages individual eligible professionals (EPs) and group practices to report information on the quality of care to Medicare. PQRS gives participating EPs and group practices the opportunity to assess the quality of care they provide to their patients, helping to ensure that patients get the right care at the right time. The Value Modifier provides for differential payment to a physician or group of physicians under the Medicare Physician Fee Schedule (PFS) based upon the quality of care furnished compared to the cost of care during a performance period. The Value Modifier is an adjustment made to Medicare payments for items and services under the Medicare PFS.

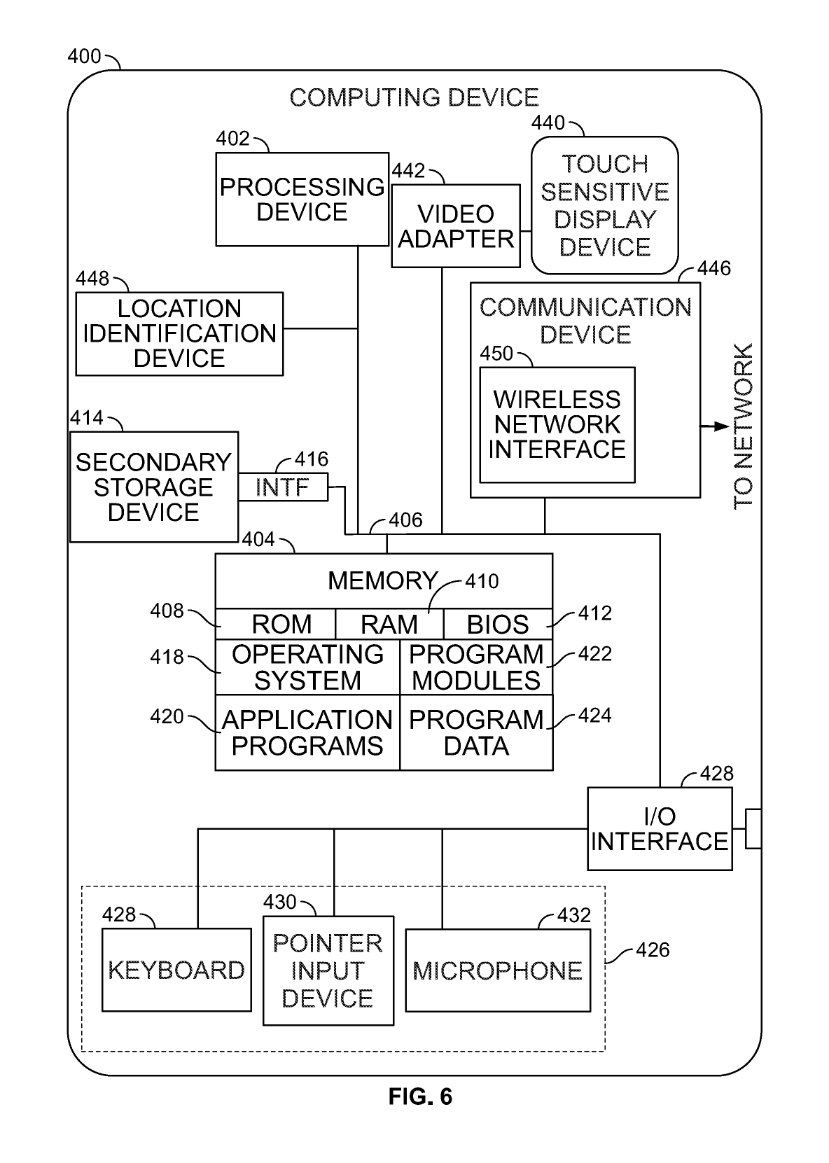

FIG. 6 illustrates an exemplary architecture of a computing device 400 which can be used to implement aspects of the present disclosure, including the patient data management system 102, the patient computing device 106, and the practitioner computing device 124, and will be referred to herein as the computing device 400. The computing device 400 is used to execute the operating system, application programs, and software modules (including the software engines) described herein.

The computing device 400 can be of various types. In some embodiments, the computing device 400 is one or more desktop computers, one or more laptop computers, other devices configured to process digital instructions, or any combination thereof. In other embodiments, the computing device 400 is one or more mobile computing devices. Examples of the computing device 400 as a mobile computing device include a mobile device (e.g., a smart phone and a tablet computer), a wearable computer (e.g., a smartwatch and a head-mounted display), a personal digital assistant (PDA), a handheld game console, a portable media player, a ultra-mobile PC, a digital still camera, a digital video camera, and other mobile devices.

The computing device 400 includes, in some embodiments, at least one processing device 402, such as a central processing unit (CPU). A variety of processing devices are available from a variety of manufacturers, for example, Intel or Advanced Micro Devices. In this example, the computing device 400 also includes a system memory 404, and a system bus 406 that couples various system components including the system memory 404 to the processing device 402. The system bus 406 is one of any number of types of bus structures including a memory bus, or memory controller; a peripheral bus; and a local bus using any of a variety of bus architectures.

The system memory 404 includes read only memory 408 and random access memory 410. A basic input/output system 412 containing the basic routines that act to transfer information within the computing device 400, such as during start up, is typically stored in the read only memory 408.

The computing device 400 also includes a secondary storage device 414 in some embodiments, such as a hard disk drive, for storing digital data. The secondary storage device 414 is connected to the system bus 406 by a secondary storage interface 416. The secondary storage devices and their associated computer readable media provide nonvolatile storage of computer readable instructions (including application programs and program modules), data structures, and other data for the computing device 400.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 414 or memory 404, including an operating system 418, one or more application programs 420, other program modules 422, and program data 424.

In some embodiments, the computing device 400 includes input devices to enable a user to provide inputs to the computing device 400. Examples of input devices 426 include a keyboard 428, a pointer input device 430, a microphone 432, and a touch sensitive display 440. Other embodiments include other input devices. The input devices are often connected to the processing device 402 through an input/output interface 438 that is coupled to the system bus 406. These input devices 426 can be connected by any number of input/output interfaces, such as a parallel port, serial port, game port, or a universal serial bus. Wireless communication between input devices and interface 438 is possible as well, and includes infrared, BLUETOOTH® wireless technology, 802.11a/b/g/n, cellular, or other radio frequency communication systems in some possible embodiments.

In this example embodiment, a touch sensitive display device 440 is also connected to the system bus 406 via an interface, such as a video adapter 442. The touch sensitive display device 440 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs.

In addition to the display device 440, the computing device 400 can include various other peripheral devices (not shown), such as speakers or a printer.

The computing device 400 further includes a communication device 446 configured to establish communication across the network. In some embodiments, when used in a local area networking environment or a wide area networking environment (such as the Internet), the computing device 400 is typically connected to the network through a network interface, such as a wireless network interface 450. Other possible embodiments use other wired and/or wireless communication devices. For example, some embodiments of the computing device 400 include an Ethernet network interface, or a modem for communicating across the network. In yet other embodiments, the communication device 446 is capable of short-range wireless communication. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The computing device 400 typically includes at least some form of computer-readable media. Computer readable media includes any available media that can be accessed by the computing device 400. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 400. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 6 is also an example of programmable electronics, which may include one or more such computing devices, and when multiple computing devices are included, such computing devices can be coupled together with a suitable data communication network so as to collectively perform the various functions, methods, or operations disclosed herein.

Referring again to FIG. 6, the computing device 400 can include a location identification device 448. The location identification device 448 is configured to identify the location or geolocation of the computing device 400. The location identification device 448 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

In some embodiments, the system of the present disclosure can utilize the regimen evaluation data 250 to evaluate symptoms and medication interactions. When tracking the regimen evaluation data 250 including information about prescribed medications and the patient's compliance with the medications, the system can further audit the data to identify medications with undesirable interaction possibilities. The system can operate to link symptoms and/or patients' complaints to possible medication side effects, and determine whether the prescription should be modified and/or how it should be changed. This enables healthcare providers to be alerted to these issues before any serious damage occurs, and further saves a trip to the hospital for patients.

The various examples and teachings described above are provided by way of illustration only and should not be construed to limit the scope of the present disclosure. Those skilled in the art will readily recognize various modifications and changes that may be made without following the examples and applications illustrated and described herein, and without departing from the true spirit and scope of the present disclosure.

What is claimed is:

1. A method for providing health treatment to a patient, comprising:
receiving, at a computing device, regimen data, the regimen data including a plurality of regimens associated with a plurality of diseases;
receiving, at the computing device, incentive data, the incentive data including a plurality of incentive options associated with the plurality of regimens;
obtaining a diagnosis of a disease;
selecting, using the computing device, a regimen from the regimen data based on the diagnosis;
selecting, using the computing device, an incentive option from the incentive data based on the selected regimen;
determining, using the computing device, compliance of the selected regimen; and
providing one or more incentives to the patient, the incentives identified from the plurality of incentive options.

2. The method of claim 1, further comprising requiring the patient to take certain actions to comply with the selected regimen.

3. The method of claim 2, further comprising defining a schedule for the patient to comply with the selected regimen.

4. The method of claim 1, further comprising setting thresholds to determine compliance with the selected regimen.

5. The method of claim 4, further comprising selecting the thresholds for the selected regime using one of:
individual data elements specific to the patient; or
group data elements specific to a group of patients.

6. The method of claim 4, further comprising allowing a caregiver to set up one or more notifications based upon the thresholds.

7. The method of claim 6, further comprising allowing the caregiver to set specific weights associated with the thresholds.

8. The method of claim 7, further comprising setting a separate threshold for each of the physiological measurements associated with the selected regimen.

9. The method of claim 1, further comprising providing one or more additional incentives to a caregiver, the incentives identified from the plurality of incentive options.

10. The method of claim 9, wherein the one or more additional incentives are selected from financial benefit, reimbursement, and discount.

11. A method for providing health treatment to a patient, comprising:
receiving, at a computing device, regimen data, the regimen data including a plurality of regimens associated with a plurality of diseases;
receiving, at the computing device, incentive data, the incentive data including a plurality of incentive options associated with the plurality of regimens;
obtaining a diagnosis of a disease;
selecting, using the computing device, a regimen from the regimen data based on the diagnosis;
selecting, using the computing device, an incentive option from the incentive data based on the selected regimen;
determining, using the computing device, compliance of the selected regimen; and
providing one or more incentives to a caregiver of the patient, the incentives identified from the plurality of incentive options.

12. The method of claim 11, further comprising requiring the patient to take certain actions to comply with the selected regimen.

13. The method of claim 12, further comprising defining a schedule for the patient to comply with the selected regimen.

14. The method of claim 11, further comprising setting thresholds to determine compliance with the selected regimen.

15. The method of claim 14, further comprising selecting the thresholds for the selected regime using one of:
individual data elements specific to the patient; or
group data elements specific to a group of patients.

16. The method of claim 14, further comprising allowing a caregiver to set up one or more notifications based upon the thresholds.

17. The method of claim 16, further comprising allowing the caregiver to set specific weights associated with the thresholds.

18. The method of claim 17, further comprising setting a separate threshold for each of the physiological measurements associated with the selected regimen.

19. The method of claim 11, further comprising providing one or more additional incentives to the patient.

20. A method for managing patient data, comprising:
receiving, at a computing device, patient data including a plurality of patient data elements;
setting, using the computing device, one or more thresholds on at least one of the plurality of patient data elements;
assigning, using the computing device, one or more weightings on at least two of the plurality of patient data elements; and
flagging, using the computing device, the plurality of patient data elements based on the one or more thresholds and the one or more weightings.
